(19) United States
(12) Patent Application Publication
(10) Pub. No.: US 2007/0203529 A1
(43) Pub. Date: Aug. 30, 2007

(54) Filtered feedthrough assembly

(76) Inventors: Rajesh V. Iyer, Eden Prairie, MN (US); Shawn D. Knowles, Saint Francis, MN (US)

Correspondence Address: MEDTRONIC, INC., 710 MEDTRONIC PARKWAY NE, MINNEAPOLIS, MN, 55432-9924, US

(21) Appl. No.: 11/363,642

(22) Filed: Feb. 28, 2006

(51) Int. Cl. A61N 1/375

(52) U.S. Cl. 607/37

(57) ABSTRACT

A filter apparatus for use with an implantable medical device includes a printed circuit board having a first trace connected to ground and adjacent feedthrough conductors extending through the printed circuit board. A chip capacitor is electrically connected between the adjacent feedthrough conductors, and is further electrically connected to the first trace on the printed circuit board.

### BACKGROUND OF THE INVENTION

The present invention relates to implantable medical devices. More particularly, the present invention relates to feedthrough assemblies having filtering capabilities.

Electrical feedthroughs provide a conductive path extending between the interior of a hermetically sealed container and a point outside the container. However, such feedthroughs also can provide a path for undesired electromagnetic interference (EMI) to enter the container. With implantable medical devices, this can lead to the undesired introduction of EMI to circuitry inside the device container.

Filtering can be provided using capacitors that are electrically connected to the conductive path or paths of the feedthrough. However, known designs using discoidal capacitor filters are expensive, and monolithic discoidal capacitors do not allow replacement of defective subcomponents during device fabrication. Moreover, many filtering assemblies are bulky and take up valuable space inside an implantable medical device container. Prior filtering assemblies do not readily provide a low-cost and small-sized filter assembly without compromising filtering performance.

### BRIEF SUMMARY OF THE INVENTION

The present invention provides an EMI-filtered feedthrough assembly for an implantable medical device. The assembly includes balanced line capacitors electrically connected between adjacent feedthrough conductors to provide low-pass filtering. Inductor coils are optionally connected to the capacitors to provide enhanced low-pass filtering.

### BRIEF DESCRIPTION OF THE DRAWINGS

FIG. 1 is a perspective view of a filter assembly according to the present invention.

FIG. 2 is a perspective view of a filtered feedthrough assembly.

FIG. 3 is a schematic circuit diagram of a portion of the filtered feedthrough assembly of FIG. 2.

FIG. 4 is a top view of an alternative filter assembly providing balanced feedthrough filtering.

FIG. 5 is a schematic circuit diagram of a portion of an alternative filtered feedthrough assembly utilizing inductor coils.

FIG. 6 is a schematic top view of an inductor coil for use with a filtered feedthrough assembly.

### DETAILED DESCRIPTION

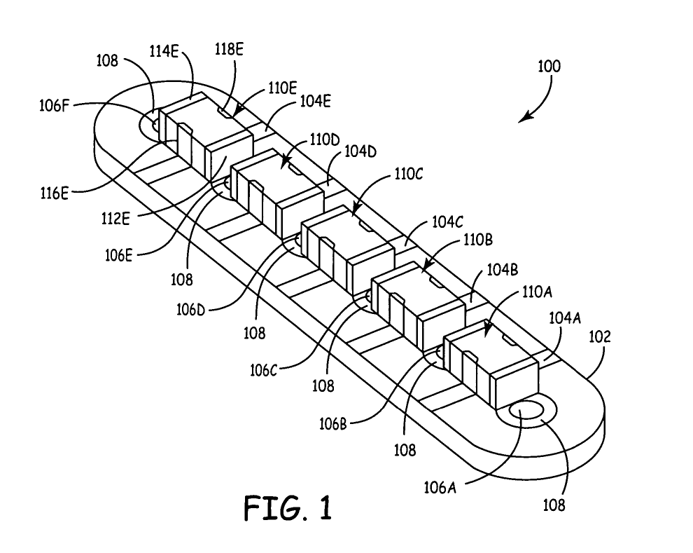

The present invention provides a filtered feedthrough assembly for an implantable medical device. FIG. 1 is a perspective view of a filter assembly 100 that includes a printed circuit board (PCB) substrate 102 with five conductive traces 104A-104E thereon. The PCB 102 can be made of a FR4 non-conductive substrate material. Six openings 106A-106F are defined through the PCB substrate 102 to permit the insertion of a feedthrough conductor (e.g., a feedthrough pin). A conductive ring 108 can optionally be disposed on the PCB substrate 102 around each opening 106A-106F, to provide mechanical reinforcement and facilitate making electrical connections at the openings 106A-106F. Each of the conductive traces 104A-104E is located between a pair of adjacent openings 106A-106F and generally extends to edges of the PCB substrate 102. In an alternative embodiment, some or all of the traces 104A-104E can be electrically connected to each other.

Five capacitors 110A-110E are each located between adjacent pairs of openings 106A-106F in the PCB substrate 102. Each capacitor is a balanced line capacitor (e.g., a balanced line capacitor available from X2Y Attenuators, LLC, Erie, Pa.), which provides increased attenuation with decreased inductance as compared to standard surface mount capacitors. As shown with respect to capacitor 110E (reference numbers for the subcomponents of capacitors 110A-110D have been omitted for clarity), each capacitor has a first connection node 112E, a second connection node 114E, a first grounding node 116E and a second grounding node 118E. The first and second grounding nodes 116E and 118E are each electrically connected to the trace 104E.

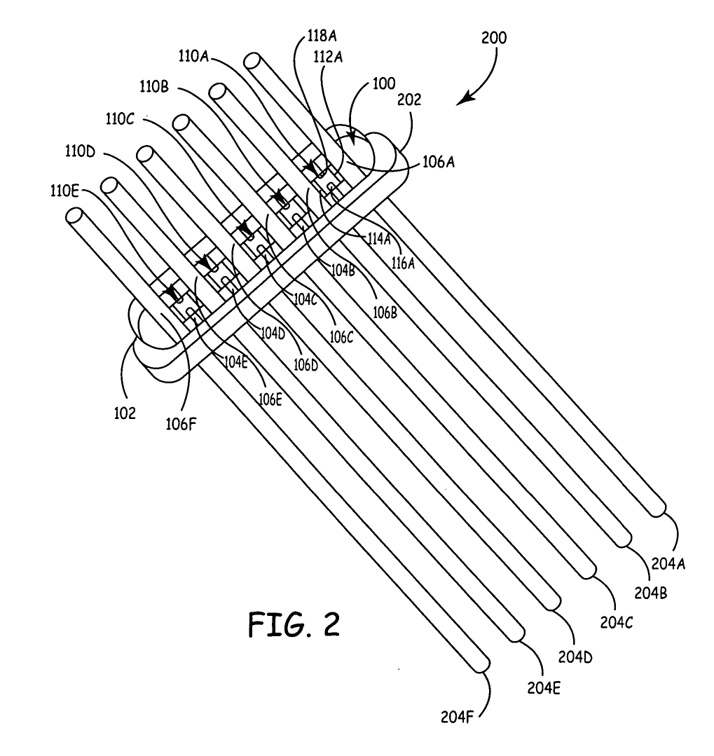

FIG. 2 is a perspective view of a filtered feedthrough assembly 200, illustrating the filter assembly 100 installed within a ferrule 202. Six feedthrough conductors 204A-204F extend through the ferrule 202 and a hermetic seal (not shown) is formed between the ferrule 202 and the feedthrough conductors 204A-204F.

The PCB substrate 102 is secured within the ferrule 202, for example, using adhesive. Typically the PCB substrate 102 has a shape that corresponds to the shape of the ferrule 202, to facilitate positioning the PCB 102 within the ferrule 202. The feedthrough conductors 204A-204F extend through the openings 106A-106F, respectively, in the PCB substrate 102.

The capacitors 110A-110E are each located between adjacent pairs of feedthrough conductors 204A-204F and mounted to the PCB substrate 102 in a conventional manner. The first connection node 112A of capacitor 110A is electrically connected to the first feedthrough conductor 204A, and the second conductor node 114A of the first capacitor is electrically connected to the second feedthrough conductor 204B. The first and second connection nodes 112B and 114B are electrically connected to the second and third feedthrough conductors 110B and 110C, respectively. The traces 104A-104E are electrically connected to the ferrule 202, which is electrically conductive and electrically grounded.

Electrical connections between components of the assembly 200 can be made using a conductive adhesive, solder, or other known techniques.

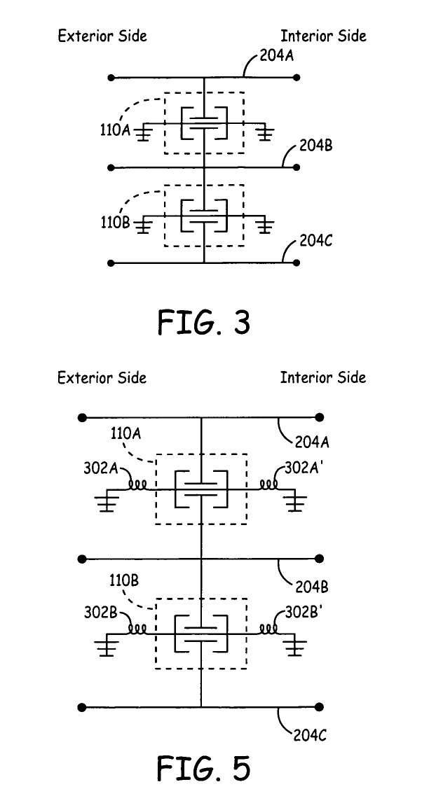

FIG. 3 is a schematic circuit diagram of a portion of the filtered feedthrough assembly 200 including three feedthrough conductors 204A-204C and two capacitors 110A and 110B. As shown in FIG. 3, each capacitor is electrically connected between adjacent feedthrough conductors in a bypass configuration, with grounding nodes of the capacitors connected to ground. Although only a portion of the assembly 200 is represented in FIG. 3, it should be recognized that the circuit can be scaled for use with any number of feedthrough conductors.

In operation, the filtered feedthrough assembly 200 provides a conductive path that can extend between an exterior side of a container and an interior side of the container. When used with an implantable medical device, electromagnetic sources in the environment may pass interference along the feedthrough. The filter assembly 100 reduces the transmission of undesired electromagnetic interference (EMI), to reduce the transmission of undesired noise while permitting desired signals to still be transmitted. The capacitors 110A-110E provide low-pass filtering. Each capacitor connected between adjacent feedthrough conductors provides simultaneous conductor-to-conductor filtering and conductor-to-ground filtering. The use of a balanced line capacitor permits this simultaneous filtering to occur without the need for separate components, thereby reducing the space occupied by the filter assembly 100.

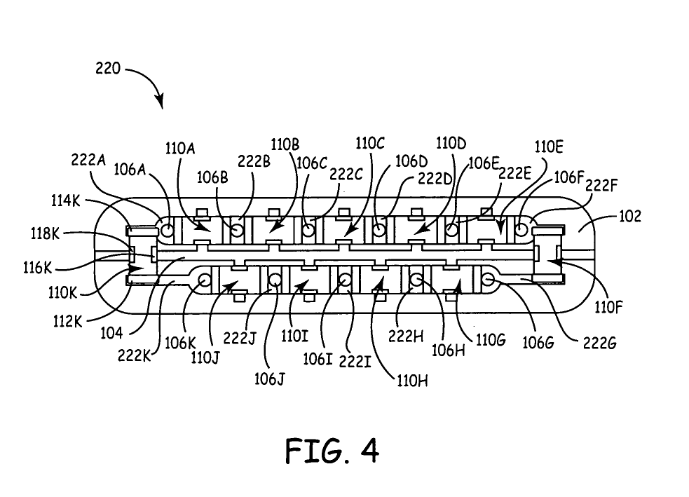

The size of each of the capacitors can vary depending on the particular application and the particular filtering desired (such as the desired cutoff frequencies). Each capacitor 110 can be of the same size. For example, each capacitor 110 can have a value of about 500 picofarads (pF) to about 10 nanofarads (nF). It is possible to provide filtering specific to each feedthrough conductor of a multipolar assembly. This can be achieved by electrically connecting only a single capacitor to particular feedthrough conductors, such as with feedthrough conductors 204A and 204F in FIG. 2. This can also be achieved by providing different sized capacitors at different locations. Alternatively, balanced filtering can be provided (see FIG. 4).

The assembly 200 provides relatively low equivalent series inductance (ESL) and equivalent series resistance (ESR) at frequencies typically involved with the design and operation of implantable medical devices.

The filter assembly 100 can be pre-fabricated and then be joined to a ferrule subassembly to form the filtered feedthrough assembly 200. This facilitates fabrication by allowing manufacture of the filter assembly 100 using conventional pick-and-place equipment to mount small components like capacitors. This avoids difficulties in mounting small capacitors directly to the filtered feedthrough assembly 200.

FIG. 4 is a top view of a filter assembly 220 that operates in a similar manner as with filter assembly 100 described above, but has an alternative configuration to provide balanced filtering. The filter assembly 220 includes a PCB substrate 102, a unitary grounding trace 104, multiple openings 106A-106K defined through the PCB substrate 102, and conductive traces 222A-222K that are each located adjacent to one of the corresponding openings 106A-106K. Balanced line chip capacitors 110A-110K having a first terminal 112A-112K, a second terminal 114A-114K, and two grounding terminals 116A-116K and 118A-118K (reference numbers for the subcomponents of the capacitors 110A-110J have been omitted for clarity). Two capacitors 110A-110K are provided for each opening 106A-106K to provide balanced filtering for feedthrough conductors positioned in the openings 106A-106K and electrically connected between adjacent conductive traces 222A-222K.

FIG. 5 is a schematic circuit diagram of an alternative embodiment of a portion of filtered feedthrough assembly 300. The assembly 300 is similar to the assembly 200 described above, but further includes an inductor coil 302 connected in series with each capacitor 110. For example, each inductor coil 302 can have a value of about 1 picohenry (pH) to about 1 nanohenry (nH), although values of the inductor coils 302 can vary according to the particular application. The assembly 300 provides an alternative filtering scheme, with the inductor coils 302 further being able to dissipate EMI. The particular electrical characteristics of the inductor coils 302A, 302A′, 302B, 302B′, as well as the characteristics of the capacitors 110A and 110B, can be selected according to the particular filtering desired for a particular application, as will be understood by those skilled in the art.

The addition of the inductor coil 302 forms an L-type filter that provides improved low frequency response of the assembly 300. More particularly, the assembly 300 has an improved attenuation slope rate as compared to the assembly 200 described above, which does not include such inductors. Thus, the use of the inductor coils 302 significantly increases the low pass filter attenuation performance of the assembly 300.

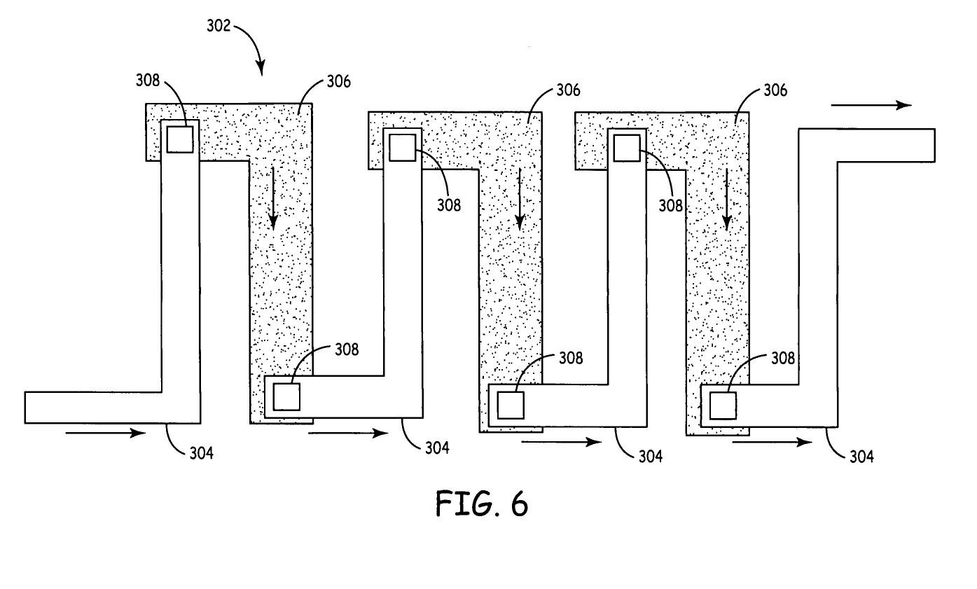

FIG. 6 is a schematic top view of an inductor coil 302, which is formed with top conductor portions 304, bottom conductor portions 306 and connectors 308 therebetween. The top and bottom conductor portions 304 and 306 are generally L-shaped, with the top and bottom portions 304 and 306 being mirror images of each other. The connectors 308 form conductive paths between the top and bottom conductor portions 304 and 306 to form the coil shape of inductor coil 302. The inductor coil 302 is typically embedded within the PCB substrate (see PCB substrate 102 in FIGS. 1 and 2), and can be formed using processes such as known deposition techniques and conventional photolithography. It should be recognized that other types of inductor coils can be used, and the inductor coil 302 shown and described with respect to FIG. 6 is merely an exemplary embodiment.

The assembly of the present invention is relatively low-cost to manufacture and occupies a relatively small space within a device, yet provides robust filtering of EMI while permitting the transmission of desired signals across the feedthrough.

Although the present invention has been described with reference to preferred embodiments, workers skilled in the art will recognize that changes may be made in form and detail without departing from the spirit and scope of the invention. For instance, the filter assemblies of the present invention can be used with a variety of feedthrough designs, including both unipolar and multipolar feedthroughs. The particular arrangement of assemblies according to the present invention will vary according to factors such as the arrangement of the feedthrough conductors.

1. A filter apparatus for use with an implantable medical device, the filter apparatus comprising:
a printed circuit board having a substrate and a first trace connected to ground;
first and second feedthrough conductors extending through the printed circuit board; and
a first chip capacitor disposed on the printed circuit board, wherein the first chip capacitor is electrically connected between the first and second feedthrough conductors, and wherein the first chip capacitor is further electrically connected to the first trace on the printed circuit board.

2. The filter apparatus of claim 1, wherein the first chip capacitor is a balanced line chip capacitor.

3. The filter apparatus of claim 1, wherein the first trace of the printed circuit board is disposed between the first and second feedthrough conductors.

4. The filter apparatus of claim 1, wherein the first trace of the printed circuit board is electrically connected to a ferrule of the implantable medical device.

5. The filter apparatus of claim 1 and further comprising:
an inductor electrically connected to the first chip capacitor.

6. The filter apparatus of claim 1 and further comprising:
a third feedthrough conductor;
a second trace on the printed circuit board connected to ground; and
a second chip capacitor electrically connected between the second and third feedthrough conductors and electrically connected to the second trace.

7. The filter assembly of claim 1, wherein a conductive adhesive is used to make electrical connections between the first chip capacitor and both the first trace and the first and second feedthrough conductors.

8. The filter assembly of claim 1 and further comprising:
a ferrule;
a hermetic seal between the first and second feedthrough conductors and the ferrule.

9. The filter assembly of claim 1, wherein the feedthrough conductors are pins.

10. A filtered feedthrough assembly comprising:
a plurality of feedthrough conductors;
a plurality of chip capacitors for providing electromagnetic interference filtering, wherein multiple chip capacitors are electrically connected to each of the feedthrough conductors, and wherein each chip capacitor is further connected to ground.

11. The feedthrough assembly of claim 10, wherein the pair of traces of the printed circuit board are grounded to the ferrule.

12. The feedthrough assembly of claim 10, wherein at least one of the plurality of feedthrough conductors is a pin.

13. The feedthrough assembly of claim 10 and further comprising:
a ferrule; and
a printed circuit board, the chip capacitors being mounted on the printed circuit board and the feedthrough conductors passing through the printed circuit board, and wherein the printed circuit board is disposed substantially within the ferrule.

14. The feedthrough assembly of claim 10, wherein the at least one chip capacitor is a balanced line chip capacitor.

15. The feedthrough assembly of claim 10, wherein at least one chip capacitors is physically located between adjacent feedthrough conductors.

16. The feedthrough assembly of claim 10 and further comprising:
an inductor electrically connected to one of the chip capacitors.

17. The feedthrough assembly of claim 10, wherein each chip capacitor is connected to ground at a pair of grounding connection points.

18. An assembly for an implantable medical device, the assembly comprising:
a feedthrough including a ferrule, first and second feedthrough pins and a hermetic seal between the ferrule and the first and second feedthrough pins;
a printed circuit board located adjacent to the ferrule, wherein the feedthrough pins extend through the printed circuit board, and wherein the printed circuit board has at least a pair of traces connected to ground; and
a balanced line chip capacitor having opposing first and second end terminals and a pair of grounding connection points connected to the pair of traces on the printed circuit board, wherein the balanced line chip capacitor is located between the first and second feedthrough pins, and wherein the first end terminal is electrically connected to the first feedthrough pin and the second end terminal is electrically connected to the second feedthrough pin.

19. The assembly of claim 18, wherein the printed circuit board is disposed substantially within the ferrule.

20. The assembly of claim 18 and further comprising:
an inductor embedded in the printed circuit board and electrically connected to the balanced line chip capacitor.
